(19) United States
(12) Patent Application Publication
(10) Pub. No.: US 2020/0340055 A1
(43) Pub. Date: Oct. 29, 2020

(54) USE OF TM9SF1 GENE AS TARGET IN VASCULAR DISEASES

(71) Applicant: Hubei University of Arts and Science, Hubei (CN)

(72) Inventors: Juan XIAO, Hubei (CN); Yanli HUANG, Hubei (CN); Chun MAO, Hubei (CN); Lin YANG, Hubei (CN); Jiao YIN, Hubei (CN); Xiaoming HE, Hubei (CN); Xiaofang SHEN, Hubei (CN); Wenbin DENG, Hubei (CN); Yuntao WU, Hubei (CN)

(21) Appl. No.: 16/316,278

(22) PCT Filed: Jul. 16, 2018
(86) PCT No.: PCT/CN2018/095821

§ 371 Date: Jan. 8, 2019

(30) Foreign Application Priority Data
Jul. 26, 2017 (CN) 201710622579.9

(51) Int. Cl. C12Q 1/6883; A61K 31/7105

(52) U.S. Cl. CPC C12Q 1/6883; A61K 31/7105; G01N 2800/32; C12Q 2600/158; C12Q 2600/136

(57) ABSTRACT

The present disclosure discloses use of TM9SF1 gene as a target in vascular diseases, which relates to the field of biotechnology. In the present disclosure, by means of RNA interference strategy, it is found that after endogenous TM9SF1 gene is interfered with, the expression of two important genes IL1β and IL8 related to the inflammation of HUVEC and the expression of the gene ACE1 closely related to vasoconstriction are remarkably downregulated, suggesting that TM9SF1 gene has positive regulation effect on the expression of IL1β, IL8 and ACE1 genes. By inhibiting or silencing the expression of the TM9SF1 gene, it is possible to inhibit or silence the expression of IL1β, IL8, and ACE1 genes, and further achieve the object of treating or preventing vascular diseases associated with the expression level of IL1β, IL8 and ACE1 genes.

The present disclosure claims the priority to the Chinese patent application No. 201710622579.9, filed with the Chinese Patent Office on Jul. 26, 2017 and entitled “Use of TM9SF1 gene as Target in Vascular Diseases”, which is incorporated herein by reference in its entirety.

### TECHNICAL FIELD

The present disclosure relates to the field of biotechnology, and particularly to use of TM9SF1 gene as a target in vascular diseases.

### BACKGROUND ART

Endothelial cells are monolayer cells located on the inner walls of blood vessels, which play a key role in the exertion of normal functions of the blood vessels, and their dysfunction is often closely related to vascular diseases.

Transmembrane 9 superfamily protein member 1 (TM9SF1) is an evolutionarily conserved nonaspanin, which is expressed in human tissues and multiple cell lines. The TM9SF1 protein is expressed by TM9SF1 gene. At present, there is little study on TM9SF1 protein or TM9SF1 gene, especially on their functions. As to the study on the relationship between the functions of endothelial cells and the TM9SF1 gene, the report thereof is even rarer.

### SUMMARY

A first object of the present disclosure includes, but is not limited to, providing use of TM9SF1 gene in screening for a medicament for treating or inhibiting inflammation of vascular endothelial cells.

A second object of the present disclosure includes, but is not limited to, providing use of TM9SF1 gene in screening for a medicament for inhibiting angiogenesis in tumor tissues.

A third object of the present disclosure includes, but is not limited to, providing use of TM9SF1 gene in screening for a medicament for treating hypertension.

A fourth object of the present disclosure includes, but is not limited to, providing use of an agent inhibiting or silencing TM9SF1 gene expression in the preparation of a medicament associated with inflammation of vascular endothelial cells, angiogenesis and hypertension.

A fifth object of the present disclosure includes, but is not limited to, providing a medicament for treating or inhibiting inflammation of vascular endothelial cells, for inhibiting angiogenesis in tumor tissues, or for treating hypertension.

A sixth object of the present disclosure includes, but is not limited to, providing a method, the method being a method of treating or inhibiting inflammation of vascular endothelial cells, a method of inhibiting angiogenesis in tumor tissues, or a method of treating hypertension.

The present disclosure is implemented as follows:

use of TM9SF1 gene as a target in screening for a medicament for treating or inhibiting inflammation of vascular endothelial cells;

use of an agent inhibiting or silencing TM9SF1 gene expression in the preparation of a medicament for treating or inhibiting inflammation of vascular endothelial cells;

use of TM9SF1 gene as a target in screening for a medicament for inhibiting angiogenesis in tumor tissues;

use of an agent inhibiting or silencing TM9SF1 gene expression in the preparation of a medicament for inhibiting angiogenesis in tumor tissues;

use of TM9SF1 gene as a target in screening for a medicament for treating hypertension;

use of an agent inhibiting or silencing TM9SF1 gene expression in the preparation of a medicament for treating hypertension;

a medicament having one or more of the following uses:

(1) treating or inhibiting inflammation of vascular endothelial cells;

(2) inhibiting angiogenesis in tumor tissues; and

(3) treating hypertension;

the medicament containing an agent that inhibits or silences TM9SF1 gene expression and pharmaceutically acceptable excipients; and

a method, the method being a method of treating or inhibiting inflammation of vascular endothelial cells, a method of inhibiting angiogenesis in tumor tissues, or a method of treating hypertension, and the method comprising administering the above-described medicament to a subject.

### The Present Disclosure Includes Following Advantageous Effects

In the present disclosure, human umbilical vein endothelial cells (HUVEC) are taken as the object of study, by means of RNA interference strategy, it is found that after endogenous TM9SF1 gene is interfered with, the expression of two important genes IL1β and IL8 related to the inflammation of HUVEC and the expression of the gene ACE1 closely related to vasoconstriction are remarkably downregulated, suggesting that TM9SF1 gene has positive regulation effect on IL1β, IL8 and ACE1 gene expression. By inhibiting or silencing the expression of the TM9SF1 gene, it is possible to inhibit or silence the expression of IL1β, IL8, and ACE1 genes, and further achieve the object of treating or preventing vascular diseases associated with the expression levels of IL1β, IL8 and ACE1 genes.

Based on this, the TM9SF1 gene can be used as a new target in screening for a medicament for treating or inhibiting inflammation of vascular endothelial cells (associated with IL1β gene expression), a medicament for inhibiting angiogenesis in tumor tissues (associated with IL8 gene expression), a medicament for treating hypertension (associated with ACE1 gene expression), and other fields.

In addition, an agent that inhibits or silences TM9SF1 gene expression can be used in the preparation of a medicament for treating or inhibiting inflammation of vascular endothelial cells, a medicament for inhibiting angiogenesis in tumor tissues, a medicament for treating hypertension, and other fields.

The new use of the TM9SF1 gene as a target in vascular diseases provided by the present disclosure provides new idea and means for treating and preventing vascular diseases.

### BRIEF DESCRIPTION OF DRAWINGS

In order to more clearly illustrate the technical solutions of the embodiments of the present disclosure, brief description is made below on the drawings required and used in the embodiments. It should be understood that the following drawings only illustrate some of the embodiments of the present disclosure and shall not be regarded as a limitation to the scope, and for a person of ordinary skills in the art, other related drawings may be obtained from these drawings without inventive effort.

FIG. 1 illustrates cell culture and identification results of HUVEC according to an embodiment of the present disclosure.

FIG. 2 illustrates verification results of the interference effect of TM9SF1 specific siRNA according to an embodiment of the present disclosure.

FIG. 3 illustrates the results of relative expression levels of inflammatory genes IL1, IL8 and ACE1 in HUVEC after being transfected by specific siRNA according to an embodiment of the present disclosure.

### DETAILED DESCRIPTION OF EMBODIMENTS

In order to make the objects, technical solutions and advantages of the embodiments of the present disclosure clearer, the technical solutions of the embodiments of the present disclosure will be described clearly and completely below. Embodiments are carried out in accordance with conventional conditions or conditions recommended by the manufacturer if no specific conditions are specified in the embodiments. Reagents or instruments used, whose manufacturers are not specified, are all conventional products that are available commercially.

Below, the use of TM9SF1 gene as a target in vascular diseases as provided in one or more embodiments of the present disclosure is specifically described.

TM9SF1 gene was cloned in 1997 for the first time. Up to now, there have been very few reports on this gene, most of which are the study on the expression thereof, and as to the study on the functions thereof, few literatures can be searched out. It has been reported that the TM9SF1 gene could induce autophagy in HeLa cells, but no relevant mechanism has been revealed. The biological functions of the TM9SF1 gene for other cells than HeLa have not been reported.

RNA interference technology is a molecular biological technique that has been widely used in recent years, and is of great significance on the aspect of exploring gene function and gene therapy. Compared with gene overexpression, which is another strategy commonly used to study gene functions, RNA interference can more exactly reflect the real physiological state of the organism/body. SiRNA is the most commonly used tool for realizing RNA interference, which has the advantages of high efficiency, high specificity, etc.

In the present disclosure, human umbilical vein endothelial cells (HUVEC) are taken as the object of study, by means of RNA interference strategy, it is found that after endogenous TM9SF1 gene is interfered with, the expression of two important genes IL1β and IL8 related to the inflammation of HUVEC and the expression of the gene ACE1 closely related to vasoconstriction are remarkably downregulated, suggesting that TM9SF1 gene has positive regulation effect on IL1β, IL8 and ACE1 gene expression. By inhibiting or silencing the expression of the TM9SF1 gene, it is possible to inhibit or silence the expression of IL1β, IL8, and ACE1 genes, and further achieve the object of treating or preventing vascular diseases associated with the expression level of IL1β, IL8 and ACE1 genes.

Based on this, the present disclosure provides use of the TM9SF1 gene as a target in the following aspects.

In a first aspect, the present disclosure provides use of the TM9SF1 gene as a target in screening for a medicament for treating or inhibiting inflammation of vascular endothelial cells.

Further, with the TM9SF1 gene as a target, the above-described medicament inhibits or silences the expression of the TM9SF1 gene.

Vascular endothelial cells are located between plasma and vascular tissues, which not only can complete metabolic exchange of plasma and tissue fluid, but also can synthesize and secrete a variety of bioactive substances to ensure the normal contraction and relaxation of blood vessels. At present, endothelial cells are known to be inflammatory cells, and their inflammatory state has significant impact on the occurrence and development of the pathophysiological processes of a variety of diseases, such as atherosclerosis, aneurysm and diabetic angiopathies. In the course of inflammation, endothelial cells can produce a variety of proinflammatory cytokines, which plays a crucial role in the further development of inflammation. For example, extracellular IL1β can activate endothelial cells (NHEK S, CLANCY R, LEE K A, et al. Activated Platelets Induce Endothelial Cell Activation via an Interleukin-1beta Pathway in Systemic Lupus Erythematosus[J]. Arterioscler Thromb Vasc Biol, 2017, 37(4):707-716. NYMO S, GUSTAVSEN A, NILSSON P H, et al. Human Endothelial Cell Activation by Escherichia coli and Staphylococcus aureus Is Mediated by TNF and IL-1beta Secondarily to Activation of C5 and CD14 in Whole Blood[J]. J Immunol, 2016, 196(5):2293-2299. Du L, DONG F, GUO L, et al. Interleukin-1beta increases permeability and upregulates the expression of vascular endothelial-cadherin in human renal glomerular endothelial cells[J]. Mol Med Rep, 2015, 11(5):3708-3714.), and endothelial cells can also produce IL1β after some stimulation, which plays an important role in the inflammatory response of endothelial cells (XIA X, SHI Q, SONG X, et al. Tetrachlorobenzoquinone Stimulates NLRP3 Inflammasome-Mediated Post-Translational Activation and Secretion of IL-1beta in the HUVEC Endothelial Cell Line[J]. Chem Res Toxicol, 2016, 29(3):421-429). Therefore, trying to inhibit the expression of IL1β in endothelial cells is of great significance for the inhibition of the inflammatory response of endothelial cells.

It has been found by study in the present disclosure that after endogenous TM9SF1 gene is interfered with, the expression level of HUVEC IL1β is significantly inhibited, suggesting that TM9SF1 may play a positive regulation role in the inflammation course of endothelial cells.

Thus, the TM9SF1 gene can be used as a target for screening for a medicament for treating or inhibiting inflammation of vascular endothelial cells. With the TM9SF1 gene as a target, by inhibiting or silencing the expression of the TM9SF1 gene, the screened medicament indirectly realizes the inhibition of the expression level of the IL1β gene, and achieves the effect of treating or inhibiting the inflammation of vascular endothelial cells.

Further, the use described above comprises:

culturing a biological sample containing TM9SF1 gene in the presence of a candidate agent;

culturing the biological sample containing TM9SF1 gene in the absence of the candidate agent; and

determining the IL1β expression levels of the above-described biological sample in the presence of the candidate agent and in the absence of the candidate agent, wherein a result that the IL1β expression level in the presence of the candidate agent is lower than the IL1β expression level in the absence of the candidate agent is an indication of the candidate agent serving as a medicament for treating or inhibiting inflammation of vascular endothelial cells.

In the above, the candidate agent inhibits or silences the expression level of TM9SF1 gene, with the TM9SF1 gene as a target.

Further, in some embodiments of the present disclosure, the candidate agent may be siRNA for the TM9SF1 gene; and may also be an antibody against TM9SF1 protein, which may inhibit the activity or reduce quantity of TM9SF1 proteins at protein level.

Further, in some embodiments of the present disclosure, the biological sample described above may be human umbilical vein endothelial cells or murine umbilical vein endothelial cells.

In a second aspect, the present disclosure provides use of an agent inhibiting or silencing TM9SF1 gene expression in the preparation of a medicament for treating or inhibiting inflammation of vascular endothelial cells.

Based on the above findings, an agent that inhibits or silences TM9SF1 gene expression can be used in the preparation of a medicament for treating or inhibiting inflammation of vascular endothelial cells, which is a new use, providing new idea and means for treating or inhibiting inflammation of vascular endothelial cells.

Further, the agent described above is siRNA for the TM9SF1 gene.

Further, the base sequence of the siRNA is as follows:

(SEQ ID NO. 1)
5′-GGUUACGACCUGACGAGUUTT-3′.

Two “TT” bases (underlined) at its 3′-end are used for improving the stability of the siRNA, and bases at positions 1-19 are used for interacting with the target gene.

SiRNA having the sequence shown in SEQ ID NO. 1 can effectively inhibit TM9SF1 gene expression. In the cells of transfected with the siRNA, the relative expression level of TM9SF1 gene is (0.11±0.04, (P<0.005)), which is far lower than that of a control group, with an interference efficiency thereof being greater than 50%. Meanwhile, the expression level of IL1β gene is (0.30±0.09, (P<0.001)), which is significantly lower than that of the control group. It is indicated that the siRNA has a relatively high interference efficiency, and can also be used as a brand-new medicament for treating or inhibiting inflammation of vascular endothelial cells.

In a third aspect, the present disclosure provides use of TM9SF1 gene as a target for screening for a medicament for inhibiting angiogenesis in tumor tissues.

Further, the medicament inhibits or silences the expression of TM9SF1 gene, with the TM9SF1 gene as a target.

One of the important cells from which IL8 is derived is endothelial cells, and IL8 itself is also one of the important participants in inflammation of endothelial cells (BORGES L E, BLOISE E, DELA C C, et al. Urocortin 1 expression and secretion by human umbilical vein endothelial cells: In vitro effects of interleukin 8, interferon gamma, lipopolysaccharide, endothelin 1, prostaglandin F-2alpha, estradiol, progesterone and dexamethasone [J]. Peptides, 2015, 74:64-69.). IL8 has a promoting effect on migration of endothelial cells (JU L, ZHOU Z, JIANG B, et al. Autocrine VEGF and IL-8 Promote Migration via Src/Vav2/Rac1/PAK1 Signaling in Human Umbilical Vein Endothelial Cells [J]. Cell Physiol Biochem, 2017, 41(4):1346-1359.) and angiogenesis in tumor tissues. Inhibition of IL8 expression can lead to inhibition of angiogenesis in tumor tissues (AALINKEEL R, NAIR B, CHEN C K, et al. Nanotherapy silencing the interleukin-8 gene produces regression of prostate cancer by inhibition of angiogenesis [J]. Immunology, 2016, 148(4):387-406. MATSUO Y, OCHI N, SAWAI H, et al. CXCL8/IL-8 and CXCL12/SDF-1alpha co-operatively promote invasiveness and angiogenesis in pancreatic cancer [J]. Int J Cancer, 2009, 124(4):853-861.).

It has been found by study in the present disclosure that after endogenous TM9SF1 is interfered with, the expression level of IL8 in HUVEC is significantly reduced, suggesting, from the opposite side, that TM9SF1 can promote the expression of IL8 in HUVEC cells. Thus, the TM9SF1 gene can be used as a target for screening for a medicament for inhibiting angiogenesis in tumor tissues. With the TM9SF1 gene as a target, by inhibiting or silencing the expression of the TM9SF1 gene, the screened medicament indirectly realizes the inhibition of the expression level of IL8 gene, and achieves the effect of inhibiting angiogenesis in tumor tissues.

Further, the use comprises:

culturing a biological sample containing TM9SF1 gene in the presence of a candidate agent;

culturing the biological sample containing TM9SF1 gene in the absence of the candidate agent; and

determining the IL8 expression levels of the above-described biological sample in the presence of the candidate agent and in the absence of the candidate agent, wherein a result that the IL8 expression level in the presence of the candidate agent is lower than the IL8 expression level in the absence of the candidate agent is an indication of the candidate agent serving as a medicament for inhibiting angiogenesis in tumor tissues.

In the above, the candidate agent inhibits or silences the expression level of TM9SF1 gene, with the TM9SF1 gene as a target.

The cDNA sequence of the TM9SF1 gene is as shown in SEQ ID NO. 2.

Further, in some embodiments of the present disclosure, the candidate agent may be siRNA for the TM9SF1 gene; and may also be an antibody against TM9SF1 protein, which may inhibit the activity or reduce quantity of TM9SF1 proteins at protein level.

Further, in some embodiments of the present disclosure, the above-described biological sample may be human umbilical vein endothelial cells or murine umbilical vein endothelial cells.

In a fourth aspect, the present disclosure provides use of an agent inhibiting or silencing TM9SF1 gene expression in the preparation of a medicament for inhibiting angiogenesis in tumor tissues.

Based on the above findings, an agent that inhibits or silences TM9SF1 gene expression can be used in the preparation of a medicament for inhibiting angiogenesis in tumor tissues. As a new use, the agent that inhibits or silences TM9SF1 gene expression provides new idea and means for inhibiting angiogenesis in tumor tissues.

Further, the agent is siRNA for the TM9SF1 gene.

Further, the base sequence of the siRNA is as follows:

(SEQ ID NO. 1)
5′-GGUUACGACCUGACGAGUUTT-3′.

SiRNA having the sequence shown in SEQ ID NO. 1 can effectively inhibit TM9SF1 gene expression. In the cells transfected with the siRNA, the relative expression level of TM9SF1 gene is (0.11±0.04, (P<0.005)), which is far lower than that of a control group, with an interference efficiency thereof being greater than 50%. Meanwhile, the expression level of IL8 gene is (0.23±0.17, (P<0.005)), which is significantly lower than that of the control group. It is indicated that the siRNA has a relatively high interference efficiency, and can also be used as a brand-new medicament for inhibiting angiogenesis in tumor tissues.

In a fifth aspect, the present disclosure provides use of TM9SF1 gene as a target for screening for a medicament for treating hypertension.

Further, the medicament described above inhibits or silences the expression of TM9SF1 gene, with the TM9SF1 gene as a target.

ACE1, also named CD143, is an important molecule leading to vasoconstriction and elevation of blood pressure. Thus, angiotensin-converting enzyme inhibitor (ACE1) is used clinically as a first-line drug for the treatment of hypertension (CHIEN S C, OU S M, SHIH C J, et al. Comparative Effectiveness of Angiotensin-Converting Enzyme Inhibitors and Angiotensin II Receptor Blockers in Terms of Major Cardiovascular Disease Outcomes in Elderly Patients: A Nationwide Population-Based Cohort Study [J]. Medicine (Baltimore), 2015, 94(43):e1751. KANDA D, TAKUMI T, MIYATA M, et al. Angiotensin-Converting Enzyme Inhibitor Prevents the Worsening of Renal Function in the Late Phase after Percutaneous Coronary Intervention [J]. J Atheroscler Thromb, 2016, 23(2):233-240. SHIH C J, CHEN H T, CHAO P W, et al. Angiotensin-converting enzyme inhibitors, angiotensin II receptor blockers and the risk of major adverse cardiac events in patients with diabetes and prior stroke: a nationwide study [J]. J Hypertens, 2016, 34(3):567-574, 575.). The results of the study suggest that after endogenous TM9SF1 is interfered with, the expression level of HUVEC ACE1 decreases by 90% or more, indicating that TM9SF1 plays an important role in promoting the expression of ACE1 in endothelial cells.

Thus, the TM9SF1 gene can be used as a target for screening for a medicament for treating hypertension. With the TM9SF1 gene as a target, by inhibiting or silencing the expression of the TM9SF1 gene, the screened medicament indirectly realizes the inhibition of the expression level of ACE1 gene, and achieves the effect of treating hypertension, i.e., lowering blood pressure.

Further, the use comprises:

culturing a biological sample containing TM9SF1 gene in the presence of a candidate agent;

culturing the biological sample containing TM9SF1 gene in the absence of the candidate agent; and

determining the ACE1 expression levels of the above-described biological sample in the presence of the candidate agent and in the absence of the candidate agent, wherein a result that the ACE1 expression level in the presence of the candidate agent is lower than the ACE1 expression level in the absence of the candidate agent is an indication of the candidate agent serving as a medicament for lowering blood pressure.

In the above, the candidate agent inhibits or silences the expression level of TM9SF1 gene, with the TM9SF1 gene as a target.

Further, in some embodiments of the present disclosure, the candidate agent may be siRNA for the TM9SF1 gene; and may also be an antibody against TM9SF1 protein, which may inhibit the activity or reduce quantity of TM9SF1 proteins at protein level.

Further, in some embodiments of the present disclosure, the above-described biological sample may be human umbilical vein endothelial cells or murine umbilical vein endothelial cells.

In a sixth aspect, the present disclosure provides use of an agent inhibiting or silencing TM9SF1 gene expression in the preparation of a medicament for treating hypertension.

Based on the above findings, an agent that inhibits or silences TM9SF1 gene expression can be used in the preparation of a medicament for treating hypertension. As a new use, the agent that inhibits or silences TM9SF1 gene expression provides new idea and means for treating hypertension.

Further, the agent is siRNA for the TM9SF1 gene.

Further, the base sequence of the siRNA is as follows:

(SEQ ID NO. 1)
5′-GGUUACGACCUGACGAGUUTT-3′.

SiRNA having the sequence shown in SEQ ID NO. 1 can effectively inhibit TM9SF1 gene expression. In the cells transfected with the siRNA, the relative expression level of TM9SF1 gene is (0.11±0.04, (P<0.005)), which is far lower than that of a control group, with an interference efficiency thereof being greater than 50%. Meanwhile, the expression level of ACE1 gene is (0.07±0.01, (P<0.001)), which is significantly lower than that of the control group. It is indicated that the siRNA has a relatively high interference efficiency, and can also be used as a brand-new medicament for lowering blood pressure.

Based on the contents of the above aspects, in the sixth aspect, the present disclosure further provides a medicament having one or more of the following uses:

(1) for treating or inhibiting inflammation of vascular endothelial cells;

(2) for inhibiting angiogenesis in tumor tissues; and

(3) for treating hypertension;

wherein the medicament contains an agent that inhibits or silences TM9SF1 gene expression and pharmaceutically acceptable excipients.

Further, in some embodiments of the present disclosure, the above-described agent is siRNA for the TM9SF1 gene; or the above-described agent is an antibody against TM9SF1 protein, which may inhibit the activity or reduce quantity of TM9SF1 proteins at protein level.

Further, the base sequence of the above-described siRNA is as follows:

(SEQ ID NO. 1)
5′-GGUUACGACCUGACGAGUUTT-3′.

Based on the contents of the above aspects, in the seventh aspect, the present disclosure further provides a method, the method being a method of treating or inhibiting inflammation of vascular endothelial cells, a method of inhibiting angiogenesis in tumor tissues, or a method of treating hypertension, and the method comprising administering to a subject the medicament provided in the sixth aspect.

Further, in some embodiments of the present disclosure, the subject described above is a mouse, a monkey or a human being.

In summary, the new uses of the TM9SF1 gene as a target and an agent inhibiting the TM9SF1 gene in vascular diseases as provided by the present disclosure provides new idea and means for treating and preventing vascular diseases.

The features and properties of the present disclosure are described in further detail below in conjunction with the examples.

### Example 1

1. Materials and Methods

1.1 Cells and Main Reagents

HUVEC was purchased from Shanghai Cell Bank of Chinese Academy of Sciences. TM9SF1 specific siRNA (SEQ ID NO. 1) was designed and synthesized by GenePharma company. The culture medium EGM specialized for endothelial cells was produced by LONZA company of Switzerland; pancreatin (containing EDTA), fetal bovine serum (FBS), phosphate buffer (PBS), etc. were produced by Hyclone company of the United States; the transfection reagent Lipofectamine 3000 was purchased from Thermo company of the United States; CD31 antibody was purchased from Immunoway company of the United States; immunocytochemical chromogenic kit was purchased from Beijing Zhongshan Golden Bridge Biotechnology Co., Ltd.; CYBR Green Mix was purchased from Beijing ComWin Biotech Co., Ltd.; and CCK8 was purchased from Yeasen Biotech Co., Ltd.

1.2 Cell Culture and Identification of HUVEC

HUVEC was cultured in the culture medium EGM specialized for endothelial cells, in an incubator at 37.5° C. and with 5% CO₂ and saturate humidity, the fluid was changed every other day, passage was carried out when the cell confluence reached 80%, the cells were digested with 0.25% pancreatin, digestion was stopped with FBS when the cells were rounded and were partially detached from the culture dish, followed by pipetting and well-mixing, then the cells were centrifuged for 5 min at 1200 r/min, the precipitate was resuspended in a culture medium, and the cells were counted and then inoculated to a new culture dish. The cells were identified by the immunocytochemical method, were fixed with 4% paraformaldehyde for 30 min when HUVEC cell confluence on cover slip reached 80%, and washed with PBS and then perforated with 0.1% Triton X-100 for 30 min. The cells were incubated overnight at 4° C. with rabbit anti-CD31 (Immunoway company, YT0752, diluted at 1:200) after blocked with goat serum for 30 min. After being washed with PBS, the cells were incubated with secondary antibody working fluid at 37° C. for 30 min, washed thoroughly with PBS, then subjected to color development with DAB for 1 min and counterstaining with hematoxylin for 1 min, and then returned blue with tap water, and mounted with neutral balsam. The resultant product was observed and photographed under an upright microscope. CD31 was mainly located on the surface of the cell membrane, and the cells with positive expression were brown in staining.

1.3 Grouping of HUVEC and Interference of TM9SF1 Gene

The cells were inoculated into a 6-well plate (5×10⁵ cells/well), were divided into a negative control group and an interference group for transfection after the cells were attached to the wall the next day. The process of transfection with Lipofectamin 3000 was as follows: 2.5 μL Lipofectamin 3000 was diluted and mixed well in 50 μL PBS, 2.5 μL siRNA (SEQ ID NO. 1) (20 μM) was diluted and mixed well in 50 μL PBS, two resulting dilutions were mixed gently and left for 5 min at room temperature, and the mixed solution was then gently dripped into the culture medium and mixed well. The solution was replaced 4-6 hours after transfection.

1.4 Real-Time Fluorescence Quantitative PCR (qPCR)

Forty-eight hours after the cells were transfected with TM9SF1 specific siRNA (SEQ ID NO. 1), the cells were lysed with Trizol to extract total RNA, 1-3 μg of the extract was subjected to reverse transcription into cDNA. With the cDNA as a template, the expression level of related genes was detected by real-time quantitative PCR using SBRY Green dye method. The amplification conditions of qPCR were as follows: 95° C. for 10 min, 95° C. for 15 s, 60° C. for 1 min, for 40 cycles in total. GAPDH was used as internal reference. The sequences of upstream and downstream primers of the genes were as follows:

| Gene names | Primer sequences |
|---|---|
| TM9SF | 5′-GCACCCTGTCGCACCAAG-3′ |
| | 5′-GACAAAGAAGAGGATGCCGTAC-3′ |
| IL1β | 5′-ACGAATCTCCGACCACCACTA-3′ |
| | 5′-CACATAAGCCTCGTTATCCCA-3′ |
| IL8 | 5′-TTGCCAAGGAGTGCTAAAGAAC-3′ |
| | 5′-CAACCCTACAACAGACCCACAC-3′ |
| ACE1 | 5′-AGGACGGCTTCACAGACACG-3′ |
| | 5′-CCAGCAGATGAGCAGGGATG-3′ |
| GAPDH | 5′-GAAGGTCGGAGTCAACGGATT-3′ |
| | 5′-CCTGGAAGATGGTGATGGGAT-3′ |

1.5 Statistical Method

The data were analyzed and processed by Graph Pad Prism 5 software. The measurement data were expressed as mean±standard deviation. The comparison between the two groups was made using an unpaired t-test, with P<0.05 meaning that the difference was of statistical significance.

2. Results

2.1 Identification of Human Umbilical Vein Endothelial Cells

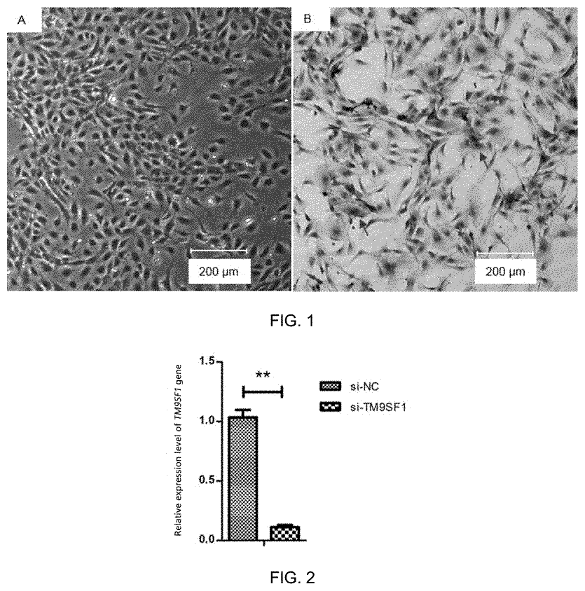

HUVEC was observed, under a phase contrast microscope, to have a good growth state, as shown in FIG. 1 (in which A showed the growth state of HUVEC observed under the phase contrast microscope; and B showed the expression of CD31 detected by immunocytochemistry, wherein the arrows indicated typical cells with strongly positive expression). The cells were polygonal, some of the cells having a few protrusions (FIG. 1-A). Since CD31 was a marker molecule of endothelial cells and mainly expressed on cell membranes, the expression of CD31 in HUVEC cells was detected by immunocytochemistry. The results showed that almost all the cells were brown, and the difference therebetween merely lies in the degree of staining, suggesting that most of the cells were positive in CD31 expression, and were typical endothelial cells (FIG. 1-B).

2.2 Verification of Interference Effect of TM9SF1 Specific siRNA

The relative expression level of TM9SF1 gene after transfection with TM9SF1 specific siRNA (SEQ ID NO. 1) was detected by qPCR technique, and the results were as shown in FIG. 2 (in which si-NC represented a negative control group, and si-TM9SF1 represented an interference group with siRNA transfection, ** indicating P<0.005). Forty-eight hours after transfection, with the negative control group (si-NC) being the reference standard 1, the relative expression level of TM9SF1 gene in the si-TM9SF1 group was (0.11±0.04, (P<0.005)), and the interference efficiency was greater than 50%, indicating that siRNA was effective.

2.3 Inhibition of the Expression of Inflammation-Related Genes in HUVEC Via Interference of TM9SF1

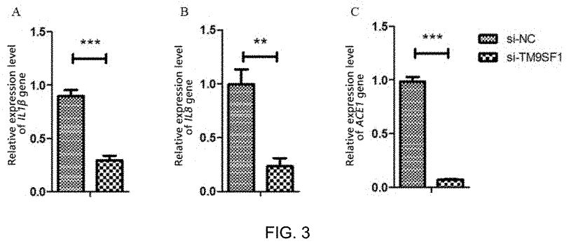

The results were as shown in FIG. 3 (in which A was the relative expression level of IL1β gene; B was the relative expression level of IL8 gene; C was the relative expression level of ACE1 gene; **P<0.005 or ***P<0.001). With the negative control group being the reference standard 1, the relative expression levels of IL1β, IL8 and ACE1 in the interference group were (0.30±0.09, (P<0.001)), (0.23±0.17, (P<0.005)) and (0.07±0.01, P<0.001)), respectively, suggesting that the expression levels were significantly inhibited.

In the present disclosure, the TM9SF1 specific siRNA was used to interfere with the endogenous expression of the gene, the interference effect thereof was verified by the real-time quantitative PCR technique, and the expression of the genes IL1, IL8 and ACE1 that were closely related to the functions of endothelial cells were found to be significantly decreased (P<0.005). These results suggested that TM9SF1 gene may play an important role in regulating the functions of endothelial cells.

This suggests that the TM9SF1 gene can be used as a new target in screening for a medicament for treating or inhibiting inflammation of vascular endothelial cells (associated with IL1β gene expression), a medicament for inhibiting angiogenesis in tumor tissues (associated with IL8 gene expression), a medicament for treating hypertension (associated with ACE1 gene expression), and other fields.

In addition, an agent (e.g., comprising siRNA shown in SEQ ID NO. 1) that inhibits or silences TM9SF1 gene expression can be used in the preparation of a medicament for treating or inhibiting inflammation of vascular endothelial cells, a medicament for inhibiting angiogenesis in tumor tissues, a medicament for treating hypertension, and other fields.

In conclusion, the new use that the TM9SF1 gene may be used as a target in vascular diseases provides new idea and means for treating and preventing vascular diseases.

The description above is merely preferred embodiments of the present disclosure, which is not used to limit the present disclosure. For a person skilled in the art, the present disclosure may have various changes and variations. Any modifications, equivalent substitutions, improvements etc. within the spirit and principle of the present disclosure shall all fall into the scope of protection of the present disclosure.

### INDUSTRIAL APPLICABILITY

The study in the present disclosure discloses that by inhibiting or silencing the expression of the TM9SF1 gene, it is possible to inhibit or silence the expression of the IL1β, IL8, and ACE1 genes. Thus, with this finding, the TM9SF1 gene can be used as a target in screening for a medicament that treats or inhibits inflammation of vascular endothelial cells, screening for a medicament for inhibiting angiogenesis in tumor tissues, screening for a medicament for treating hypertension, and other fields; and meanwhile, an agent that inhibits or silences TM9SF1 gene expression can be used in the preparation of a medicament that treats or inhibits inflammation of vascular endothelial cells, a medicament that inhibits angiogenesis in tumor tissues, a medicament that treats hypertension, and other fields.

1. A method for screening for a medicament for treating or inhibiting a vascular disease using TM9SF1 gene as a target, wherein the vascular disease is inflammation of vascular endothelial cells, angiogenesis in tumor tissues or hypertension.

2. The method according to claim 1, wherein the medicament inhibits or silences expression of TM9SF1 gene, with TM9SF1 gene as a target.

3. The method according to claim 1, wherein the vascular disease is inflammation of vascular endothelial cells, and
the method comprises:
culturing a biological sample containing TM9SF1 gene, in presence of a candidate agent;
culturing a biological sample containing TM9SF1 gene, in absence of the candidate agent; and
determining IL1β expression levels of the biological sample in the presence of the candidate agent and the biological sample in the absence of the candidate agent, wherein a result that the IL1β expression level obtained in the presence of the candidate agent is lower than the IL1β expression level obtained in the absence of the candidate agent is an indication of the candidate agent serving as the medicament for treating or inhibiting inflammation of vascular endothelial cells.

4. The method according to claim 3, wherein the candidate agent inhibits or silences an expression level of TM9SF1 gene, with TM9SF1 gene as a target.

5. The method according to claim 4, wherein the candidate agent is a siRNA for TM9SF1 gene; or
the candidate agent is an antibody against TM9SF1 proteins, which can inhibit an activity of TM9SF1 proteins or reduce quantity of TM9SF1 proteins at a protein level.

6. (canceled)

7. (canceled)

8. (canceled)

9. The method according to claim 1, wherein the vascular disease is angiogenesis in tumor tissues, and
the method comprises:
culturing a biological sample containing TM9SF1 gene in presence of a candidate agent;
culturing a biological sample containing TM9SF1 gene in absence of the candidate agent; and
determining IL8 expression levels of the biological sample in the presence of the candidate agent and the biological sample in the absence of the candidate agent, wherein a result that the IL8 expression level obtained in the presence of the candidate agent is lower than the IL8 expression level obtained in the absence of the candidate agent is an indication of the candidate agent serving as the medicament for inhibiting angiogenesis in tumor tissues.

10. The method according to claim 9, wherein the candidate agent inhibits or silences an expression level of TM9SF1 gene, with TM9SF1 gene as a target.

11. The method according to claim 10, wherein the candidate agent is a siRNA for TM9SF1 gene; or
the candidate agent is an antibody against TM9SF1 proteins, which can inhibit an activity of TM9SF1 proteins or reduce quantity of TM9SF1 proteins at a protein level.

12. (canceled)

13. (canceled)

14. (canceled)

15. The method according to claim 1, wherein the vascular disease is hypertension, and
the method comprises:
culturing a biological sample containing TM9SF1 gene in presence of a candidate agent;
culturing a biological sample containing TM9SF1 gene in absence of the candidate agent; and
determining ACE1 expression levels of the biological sample in the presence of the candidate agent and the biological sample in the absence of the candidate agent, wherein a result that the ACE1 expression level obtained in the presence of the candidate agent is lower than the ACE1 expression level obtained in the absence of the candidate agent is an indication of the candidate agent serving as the medicament for lowering a blood pressure.

16. The method according to claim 15, wherein the candidate agent inhibits or silences an expression level of TM9SF1 gene, with TM9SF1 gene as a target.

17. (canceled)

18. (canceled)

19. A medicament, having one or more of the following uses:
(1) for treating or inhibiting inflammation of vascular endothelial cells;
(2) for inhibiting angiogenesis in tumor tissues; and
(3) for treating hypertension,
wherein the medicament contains an agent that inhibits or silences TM9SF1 gene expression and pharmaceutically acceptable excipients.

20. A method, the method being a method of treating or inhibiting a vascular disease, wherein the method comprises administering to a subject the medicament of claim 19, and the vascular disease is inflammation of vascular endothelial cells, angiogenesis in tumor tissues, or hypertension.

21. The method according to claim 16, the candidate agent is a siRNA for TM9SF1 gene; or the candidate agent is an antibody against TM9SF1 proteins, which can inhibit an activity of TM9SF1 proteins or reduce quantity of TM9SF1 proteins at a protein level.

22. The method according to claim 20, wherein the agent is a siRNA for TM9SF1 gene.

23. The method according to claim 2, wherein the vascular disease is inflammation of vascular endothelial cells, and
the method comprises:
culturing a biological sample containing TM9SF1 gene, in presence of a candidate agent;
culturing a biological sample containing TM9SF1 gene, in absence of the candidate agent; and
determining IL1β expression levels of the biological sample in the presence of the candidate agent and the biological sample in the absence of the candidate agent, wherein a result that the IL1β expression level obtained in the presence of the candidate agent is lower than the IL1β expression level obtained in the absence of the candidate agent is an indication of the candidate agent serving as the medicament for treating or inhibiting inflammation of vascular endothelial cells.

24. The method according to claim 2, wherein the vascular disease is angiogenesis in tumor tissues, and
the method comprises:
culturing a biological sample containing TM9SF1 gene in presence of a candidate agent;
culturing a biological sample containing TM9SF1 gene in absence of the candidate agent; and
determining IL8 expression levels of the biological sample in the presence of the candidate agent and the biological sample in the absence of the candidate agent, wherein a result that the IL8 expression level obtained in the presence of the candidate agent is lower than the IL8 expression level obtained in the absence of the candidate agent is an indication of the candidate agent serving as the medicament for inhibiting angiogenesis in tumor tissues.

25. The method according to claim 2, wherein the vascular disease is hypertension, and
the method comprises:
culturing a biological sample containing TM9SF1 gene in presence of a candidate agent;
culturing a biological sample containing TM9SF1 gene in absence of the candidate agent; and
determining ACE1 expression levels of the biological sample in the presence of the candidate agent and the biological sample in the absence of the candidate agent, wherein a result that the ACE1 expression level obtained in the presence of the candidate agent is lower than the ACE1 expression level obtained in the absence of the candidate agent is an indication of the candidate agent serving as the medicament for lowering a blood pressure.


SEQUENCE LISTING (2 sequences)

SEQ ID NO: 1 - RNA, 19 nt, Artificial Sequence
  siRNA for TM9SF1 Gene
gguuacgaccugacgaguu

SEQ ID NO: 2 - DNA, 1620 bp, Artificial Sequence
  cDNA for TM9SF1 Gene
tttccgccaggctgaggtcgccgccggtgagggcggaagtggtaagactgacgtgtcctgggccgcgctgccgatcgccgggaggacccccgcctcgccgaagacgggcggggcaagccgagcctcacggggtccccggagctgggccgggcctccagatggagaaggcgcaacggggagttcttgagtaagccagagcggtgtccagcgcggtgtagccgcagccgccgctgtcaggcgcagcaacgggcaaccccgtagaagtcggtcggcaggtcctctccaacccgccgctaccgcgccgctgtgggagagaccccagcaggagcccaaaggcagctacgggggcgcgaaggccgctggcgccgcctcggccagcccttcccgcgcggttccactgccttaaggatgacagtcgtagggaaccctcgaagttggagctgccagtggttgccaatcctgatactgttgctgggcacaggccatgggccaggggtggaaggcgtgacacactacaaggccggcgaccctgttattctgtatgtcaacaaagtgggaccctaccataaccctcaggaaacttaccactactatcagcttccagtctgctgccctgagaagatacgtcacaaaagccttagcctgggtgaagtgctggatggggaccgaatggctgagtctttgtatgagatccgctttcgggaaaacgtggagaagagaattctgtgccacatgcagctcagttctgcacaggtggagcagctgcgccaggccattgaagaactgtactactttgaatttgtggtagatgacttgccaatccggggctttgtgggctacatggaggagagtggtttcctgccacacagccacaagataggactctggacccatttggacttccacctagaattccatggagaccgaattatatttgccaatgtttcagtgcgggacgtcaagccccacagcttggatgggttacgacctgacgagttcctaggccttacccacacttatagcgtgcgctggtctgagacttcagtggagcgtcggagtgacaggcgccgtggtgacgatggtggtttctttcctcgaacactggaaatccattggttgtccatcatcaactccatggtgcttgtgtttttactggtgggttttgtggctgtcattctaatgcgtgtgcttcggaatgacctggctcggtacaacttagatgaggagaccacctctgcaggttctggtgatgactttgaccagggtgacaatggctggaaaattatccatacagatgtcttccgcttccccccataccgtggtctgctctgtgctgtgcttggcgtgggtgcccagttcctggcccttggcactggcattattgtcatggcactgctgggcatgttcaatgtgcaccgtcatggggccattaactcagcagccatcttgttgtatgccctgacctgctgcatctctggctacgtgtccagccacttctaccggcagattggaggcgagcgttgggtgtggaacatcattctcaccaccagtctcttctctgtgcctttcttcctgacgtggagtgtggtgaactcagtgcattgggccaatggttcgaca